(19) United States
(12) Patent Application Publication
(10) Pub. No.: US 2016/0320316 A1
(43) Pub. Date: Nov. 3, 2016

(54) METHOD FOR DETERMINING THE CELL AGGRESSIVENESS GRADE OF CANCER CELLS OR OF CANCER STEM CELLS

(71) Applicants: UNIVERSITE DE LIMOGES, Limoges (FR); ONCOMEDICS, Limoges Cedex 3 (FR)

(72) Inventors: Arnaud POTHIER, CONDAT SUR VIENNE (FR); Claire JEROME, LIMOGES (FR); Pierre BLONDY, LIMOGES (FR); Fabrice LALLOUE, ISLE (FR); Serge BATTU, LIMOGES (FR); Marie-Odile JAUBERTEAU, LIMOGES (FR); Philippe CARDOT, LIMOGES (FR); Christophe LAUTRETTE, AIXE SUR VIENNE (FR); Stephanie GIRAUD, FEYTIAT (FR); Christophe MORAND DU PUCH, RILHAC-RANCON (FR)

(73) Assignees: UNIVERSITE DE LIMOGES, LIMOGES (FR); ONCOMEDICS, LIMOGES CEDEX 3 (FR)

(21) Appl. No.: 15/107,831

(22) PCT Filed: Dec. 24, 2014
(86) PCT No.: PCT/FR2014/053552

§ 371 Date: Jun. 23, 2016

(30) Foreign Application Priority Data
Dec. 24, 2013 (FR) 13 63547

(51) Int. Cl. G01N 22/00; G01N 33/483

(52) U.S. Cl. CPC G01N 22/00; G01N 33/4833

(57) ABSTRACT

Method for determining, in vitro, the cell aggressiveness grade of cancer cells or for detecting cancer stem cells in a cell sample originating from a solid tissue suspected of being cancerous, includes: a) dissociating the cell cluster constituting the sample into a suspension of whole and viable isolated cells, b) macroscopically sorting the cells to obtain homogeneous subpopulations, c) calibrating at least one microwave electromagnetic sensor resonating at its own resonance frequency, d) presenting the dissociated and sorted cells to the calibrated sensor, e) interrogating the sensor and determining its new resonance frequency having received the cells, f) calculating the variation in overall dielectric permittivity of the cells according to the variation in working frequency, which constitutes the electromagnetic signature of the cells. The macroscopic sorting is without prior labelling and is based on the intrinsic properties of the cells. A kit for implementing the method is also described.

This invention relates to a process for determining the cell aggressiveness grade of cancer cells or of cancer stem cells.

Aside from a visual examination of cells, practitioners have relatively few ways to determine the aggressiveness grade of cancer cells.

There are currently no markers linked to the aggressiveness of cancer cells, at least no markers of certainty making it possible to determine the aggressiveness of cancer cells.

Hereinafter, the term “grade” will be used for the aggressiveness level of the tumor cell and the term “stage” for the aggressiveness and organization level at the tissue level.

It is known that cancerous tumors are classified according to multiple categories based on the TNM classification, from the least aggressive tumor stage to the most aggressive tumor stage. In the case of colorectal cancer, there are five (5) stages:
Stage 0: The tumor is superficial and does not invade the submucosa; the lymphatic ganglia are not reached; there is no remote metastasis.
Stage I: The tumor invades the submucosa or the muscular layer of the wall of the colon or the rectum; the lymphatic ganglia are not reached; there is no metastasis.
Stage II: The cancer cells have passed through multiple layers of the wall of the colon or the rectum; the lymphatic ganglia are not reached; there is no remote metastasis.
Stage III: The cancer cells have invaded the lymphatic ganglia close to the tumor.
Stage IV: The cancer spreads beyond the colon or the rectum toward organs that are farther away.

The most aggressive stage is the one that corresponds to the formation of metastases.

Thus, the visual examination consists in analyzing the morphology anomalies of the cells, which is a method that is at least labor-intensive and very time-consuming, and which can in no case be automated. The resulting cost is necessarily very high. It is therefore understood that there can be a crucial need for an alternative process.

A publication “Label-Free Colorectal Cancer Line Bio-Sensing Using RF Resonator” XLIM UMR 7252 CNRS/University of Limoges, Homéostasie Cellulaire et Pathologies [Cell Homeostasis and Pathologies] EA3842, University of Limoges, ONCOMEDICS June 2013, mentions the use of resonating microwave electromagnetic micro-sensors, making it possible to determine the aggressiveness of cancer cells from values for measurement of the dielectric properties of cells using these micro-sensors.

This analysis by dielectric spectroscopy on the scale of the cell is based on the use of the difference in resonance frequency of these micro-sensors when they are devoid of any cells and when a cell or several cells rest on said micro-sensor.

It is noted that this analysis does not require any labeling of the cells in advance.

It is necessary to indicate that the electromagnetic waves of the microwave spectrum, used for interrogating the cells, lead to a discriminating result because the cancer cells under study have a high permittivity with regard to these electromagnetic waves of the microwave spectrum. Actually, the conductivity and the permittivity of a normal cell are less than that of a cancer cell.

These variations of the resonance frequency and therefore the responses of the micro-sensors are linked in particular to the size of the cells, to the volume, and to the permittivity of the intracellular contents, to the concentration of significant ions such as the potassium, sodium, and calcium ions, and to the quantity of chromatin in the core in relation to the cell volume.

Nevertheless, the difficulty of implementing the process resides in the fact that measurement using electromagnetic micro-sensors operating in a resonator requires a very limited number of cells.

However, any process for determining the aggressiveness grade of a cell aimed at industrial and commercial usage requires reproducibility, quality, and simple and fast implementation in comparison to a research laboratory process.

This is the object of this invention that proposes a process for determining the cell aggressiveness grade of cancer cells or of cancer stem cells that responds to the needs of analyses for numbers.

For this purpose, the object of the invention is a process for determining in vitro the cell aggressiveness grade of cancer cells or for detecting cancer stem cells in a cell sample originating from a solid tissue that is suspected of being cancerous, comprising at least the following steps:
a. Dissociation of the cell cluster constituting the sample into a suspension of whole and viable isolated cells,
b. Macroscopic sorting of cells to obtain homogeneous subpopulations,
c. Calibration of at least one microwave electromagnetic sensor resonating at its own resonance frequency,
d. Presentation of the cells that are dissociated and sorted according to steps a. and b. on the at least one previously calibrated sensor,
e. Interrogation of the at least one sensor and determination of the new resonance frequency of said at least one sensor having received the cells,
f. Calculation of the variation in overall dielectric permittivity of the cells based on the variation of the work frequency, which constitutes the electromagnetic signature of the cells.

The invention is now described in detail.

The process for analysis on resonating electromagnetic biosensors requires the preparation of cells from a sample of living tissue that is taken. This sample is to be preserved between 2 and 8° C., in a suitable medium, and there is known for this purpose in particular a composition marketed under the name OncoWave-Via, of the Oncomédics Company (France). Actually, it is necessary to be able to dissociate the cells so as to obtain individualized cells because the biosensors aim for measurements on the monocellular scale, and even on the scale of several cells, with the number being less than 10 to provide an order of magnitude. The dissociation step preferably consists in producing at least mechanical dissociation and enzymatic dissociation.

Mechanical dissociation consists in particular in cutting the sample taken into tissue fragments of approximately 1 to 3 mm³, preferably of a size of less than 2 mm³.

Enzymatic dissociation is preferably carried out using at least two enzymes. It can consist in immersing these fragments in a dissociation solution, such as the solution marketed under the name OncoWava-Diss, Oncomédics Company (France). Preferably, the enzymatic dissociation is carried out using at least:
Collagenase, Type II: This enzyme ensures cleavage of the peptide bonds of collagen proteins by degrading the extracellular matrix and by releasing the cells from it into the surrounding environment, and/or
Trypsin, which is an aspecific endoprotease that degrades just as well the proteins that it encounters and reinforces the action of the collagenase. This endoprotease also ensures the individualization of the cells from the possible clusters of non-individualized cells, generated by the collagenase, by cleaving the direct cell-cell bonds.

Such a dissociation is obtained in 1 to 2 hours to provide an order of magnitude.

The solution is then preferably filtered using a 40 μm cellular sieve so as to eliminate the tissue fragments that are not digested by the enzymatic action.

An inhibiting solution, in particular trypsin, makes it possible to stop the dissociation and to preserve the cells and more particularly to avoid degrading the membrane.

The filtrate is then centrifuged so as to recover the cellular cap.

It is noted that approximately 80% of the cells are thus preserved alive.

Once the cells are isolated, after the dissociation stage, it is advisable to sort the heterogeneous tumor cells based on their intrinsic physico-chemical properties, in particular the size, the density, the shape, or the deformability. It is necessary that this sorting be obtained without fluorescent or magnetic immunological labeling, able to modify the state of cellular activation.

The sorting is therefore preferably done by the SdFFF (coupling by Sedimentation Field-Flow Fractionation) method. This method and the device necessary for its implementation are fully described in the thesis of Sep. 28, 2007, Gaëlle BEGAUD, having as its subject: “Fractionnement par couplage Flux Force de Sedimentation: applications au tri cellulaire dans le domaine de l'oncologie [Coupling by Sedimentation Field-Flow Fractionation: Applications to Cell Sorting in the Field of Oncology]” pp. 84-92 and in the patent EP 1 679 124.

The cell populations preserve their viability and their integrity.

The cell fractions obtained are homogenized.

After this step b. of macroscopic sorting of cells, the process comprises a step c. of calibrating at least one microwave electromagnetic sensor resonating at its own resonance frequency.

The sensors of dielectric permittivity that are used are resonating electromagnetic biosensors of planar geometries and millimetric dimensions. These sensors are produced by employing substrates used in microelectronics, in particular 500 μm-thick silica sheets. A thin gold-metallic film, with a very slight thickness of 4 to 5 μm of thickness for specifying the range of values, defined by chemical etching, makes it possible to produce the resonating circuit, with inductance being associated in parallel with a capacitance, where the circuit is equipped with interdigitated electrodes. The gold is used for its excellent electrical conductivity, for its stability faced with oxidation, and for its biocompatibility.

The interdigitated spaces of the circuit accommodate said cells.

Biocompatible polymer coatings can be deposited on the sensors, around their electrodes so as to delimit microscopic analysis chambers designed to accommodate the cells so that they react with said sensor.

Based on the geometry of the sensor, it is advisable to determine the resonance frequency for which the biosensor has maximum power absorption of the electromagnetic waves of the microwave spectrum used that interrogates it.

It is noted that when cells are present on the biosensor, the value of this resonance frequency is modified.

The offsetting of the resonance is related to the number of cells present, to the volume of these cells, and to the dielectric properties of these cells to define a reproducible value.

The cell volume can be determined by available commercial means such as a meter of the BECKMAN Coulter brand.

By using multiple sensors operating at different frequencies, it then is possible to recreate an electromagnetic signature, of the cell type, analyzed by the sensors.

Thus, it is possible to make use of sensors having a single, fixed resonance frequency, in a range of between 1 and 40 GHz, preferably between 5 and 14 GHz. The periodicity of the measurements is on the order of 500 MHz to 1 GHz.

Nevertheless, to obtain a more precise, more complete, signature, it is possible to make use of electromagnetic sensors that have means for adjustments of their resonance frequency in a given range. Such sensors are each equipped in a known way with a tuning component, for example a diode or a variable capacitor or else a switchable capacitor bank, mounted in parallel with the capacitor of the resonator, with said tuning component being supplied with external voltage.

The frequency that is used can thus be adjusted continuously to determine the properties of the cells analyzed on a continuous spectrum of frequencies.

The determination of the properties of the cells is preferably carried out in the manner now described.

With at least one cell being deposited in the interdigitated spaces of the circuit, there is a modification of the response of the resonating sensor.

The responses of the biosensor do not make it possible to determine whether these are cytoplasm, concentrations of proteins, inherent properties of the core, or organelles that are the cause thereof, but there is a determination of the average dielectric properties of cells that have been sorted to determine a homogeneous population.

The determination of the cell permittivity is established from a mathematical model in which the cell is assimilated with a uniform dielectric particle placed between two electrodes.

Each cell thus acts as an additional capacitive element C_{cell} that increases the initial capacitive value of the sensor.

In the case of multiple cells, there is an accumulation.

The resonator LC sees its frequency vary from f₀, sensor without a cell, to sensor with at least one cell:

$${f}_{0} = \frac{1}{2 π √ {LC}_{0}}$$

$${f}_{1} = \frac{1}{2 π √ {LC}_{1}}$$

With C₁=C₀+ΣC_{cell}, C₀ and C₁ that represent the equivalent capacitances of a sensor without a cell and with a cell, it is possible to determine the total capacitance by the formula:

ΣC_{cell}=N_{cell}·C_{cell}=C₀(f₀−f₁)(f₀+f₁)/f₁²

N_{cell} represents the number of cells on the biosensor.

To take into account the volume of the cells V_{cell} and if we continue to use the model that calls for each cell to complete the space W_{IDC} between two electrodes, then the permittivity is provided by the following formula, with ε₀ being the effective permittivity of the vacuum:

ε_{cell}=(C_{cell}·W_{IDC})/(ε₀·V_{cell}·N_{cell})

The permittivity of the cell is thus determined by the following calculation formula:

ε_{cell}=C₀·W_{IDC}²((f₀−f₁)(f₀+f₁)/(f₁²·ε₀·V_{cell}·N_{cell})

This formula requires very limited calculation power compared to the one that employs simulations with finite-element calculations. The characterizations are therefore obtained more quickly and more easily than with a calculation based on a modeling by finite elements.

Therefore, it is thus possible to determine the permittivity of the cells of the same type and to obtain a specific signature corresponding to the different cell aggressiveness grades by analyzing a sample that is taken, preserved alive.

Thus, the dielectric permittivity of the cells is determined essentially from the following parameters: number of cells analyzed, volume of cells, and frequency offset between the inherent resonance frequency of the biosensor and the resonance frequency measured when the cells have been deposited in the electrodes.

It is also possible to provide analyses with sensors of different types, no longer in a static manner (cells that are deposited or kept immobilized on the biosensor) but in a dynamic manner (cells moving in a stream).

In this case, fluid microchannels are provided that make it possible to present cells individually to the biosensors. These cells are transported in a suitable support medium to the detection electrodes of the biosensors. The population is analyzed in a dynamic way in the manner of a flow cytometer.

An associated kit for determining in vitro the cell aggressiveness grade of cancer cells or for detecting cancer stem cells in a cell sample originating from solid tissue suspected of being cancerous is also provided, with said kit comprising at least:
Solutions for preserving and transporting the biological sample after sampling constituted of in particular organic and inorganic nutrients in the form of salts, amino acids, fatty acids, peptides, proteins, and lipoproteins, buffer system carbohydrates for maintaining the pH and metallic trace elements
Compositions of the medium for enzymatic dissociation of the biological sample constituted of in particular organic and inorganic nutrients in the form of salts, amino acids, fatty acids, peptides, proteins and lipoproteins, buffer system carbohydrates for maintaining the pH and metallic trace elements and enzymes
Consumables for the macroscopic cell sorting of the biological sample by coupling by Sedimentation Field-Flow Fractionation, SdFFF,
At least one biosensor
Composition for receiving cells and presenting them to at least one biosensor.

The process according to this invention thus makes it possible to determine the cell aggressiveness grade of cancer cells or to detect cancer stem cells in a cell sample originating from solid tissue, without modification of cells by a labeling, in particular without fluorescent or magnetic immunological labeling, able to modify the state of cellular activation.

1. Process for determining in vitro the cell aggressiveness grade of cancer cells or for detecting cancer stem cells in a cell sample originating from solid tissue that is suspected of being cancerous, comprising at least the following steps:
a. Dissociation of the cell cluster constituting the sample into a suspension of whole and viable isolated cells,
b. Macroscopic sorting of cells to obtain homogeneous subpopulations,
c. Calibration of at least one microwave electromagnetic sensor resonating at its own resonance frequency,
d. Presentation of the cells that are dissociated and sorted according to steps a. and b. on the at least one previously calibrated sensor,
e. Interrogation of the at least one sensor and determination of the new resonance frequency of said at least one sensor having received the cells,
f. Calculation of the variation in overall dielectric permittivity of the cells based on the variation of the work frequency, which constitutes the electromagnetic signature of the cells.

2. Determination process according to claim 1, step a. for dissociation comprises at least mechanical dissociation and enzymatic dissociation.

3. Determination process according to claim 2, wherein mechanical dissociation consists in producing tissue fragments of a size less than 2 mm³.

4. Determination process according to claim 2, wherein enzymatic dissociation is carried out with at least two enzymes.

5. Determination process according to claim 1, wherein enzymatic dissociation is carried out with collagenase and/or trypsin.

6. Determination process according to claim 1, wherein the macroscopic sorting of step b. is carried out by coupling by Sedimentation Field-Flow Fractionation.

7. Determination process according to claim 1, wherein multiple sensors working at different frequencies are used.

8. Determination process according to claim 1, wherein sensors with adjustable resonance frequency are used in such a way as to limit the number of sensors to be used.

9. Determination process according to claim 1, wherein operations are carried out at a frequency bandwidth of between 1 and 40 GHz.

10. Determination process according to claim 9, wherein operations are carried out in a frequency spectrum of between 5 and 14 GHz.

11. Determination process according to claim 1, wherein the dielectric permittivity of the cells is determined from the following parameters: number of cells analyzed, volume of cells and frequency offset between the inherent resonance frequency and the resonance frequency measured in step e.

12. Determination process according to claim 11, wherein the permittivity of a type of cell is obtained by the following formula:
ε_{cell}=C₀·W_{IDC}²(f₀−f₁)(f₀+f₁)/(f₁²·ε₀·V_{cell}·N_{cell})
with
$$\text{:} {f}_{0} = \frac{1}{2 π √ {LC}_{0}}$$
$$\text{:} {f}_{1} = \frac{1}{2 π √ {LC}_{1}}$$
$$\text{:} {C}_{1} = {C}_{0} + \sum {C}_{cell}$$
$$\text{:} \sum {C}_{cell} = {N}_{cell} \cdot {C}_{cell} = {C}_{0} ( {f}_{0} - {f}_{1} ) ( {f}_{0} + {f}_{1} ) \text{/} {f}_{1}^{2}$$
:N_{cell} represents the number of cells on the biosensor
:C₀ and C₁ represent capacitances of a sensor without and with at least one cell
: .ε₀ represents the permittivity of the vacuum.

13. Kit for determining in vitro the cell aggressiveness grade of cancer cells or for detecting cancer stem cells in a cell sample originating from solid tissue suspected of being cancerous, comprising at least:
Solutions for preserving and transporting the biological sample after sampling constituted of in particular organic and inorganic nutrients in the form of salts, amino acids, fatty acids, peptides, proteins and lipoproteins, carbohydrates of buffer systems for maintaining the pH and metallic trace elements
Compositions of the medium for enzymatic dissociation of the biological sample constituted of in particular organic and inorganic nutrients in the form of salts, amino acids, fatty acids, peptides, proteins and lipoproteins, buffer system carbohydrates for maintaining the pH and metallic trace elements and enzymes
Consumables for the macroscopic cell sorting of the biological sample by coupling by Sedimentation Field-Flow Fractionation, SdFFF,
At least one biosensor
Composition for receiving cells and presenting them to at least one biosensor.

14. Determination process according to claim 3, wherein enzymatic dissociation is carried out with at least two enzymes.

15. Determination process according to claim 2, wherein enzymatic dissociation is carried out with collagenase and/or trypsin.

16. Determination process according to claim 3, wherein enzymatic dissociation is carried out with collagenase and/or trypsin.

17. Determination process according to claim 4, wherein enzymatic dissociation is carried out with collagenase and/or trypsin.
